(19) United States
(12) Patent Application Publication
(10) Pub. No.: US 2025/0051624 A1
(43) Pub. Date: Feb. 13, 2025

(54) DUST SUPPRESSANT COMPOSITION COMPRISING GLYCOLIPID

(71) Applicant: ARIZONA BOARD OF REGENTS ON BEHALF OF THE UNIVERSITY OF ARIZONA, Tucson, AZ (US)

(72) Inventors: Minkyu Kim, Tucson, AZ (US); David Hogan, Tucson, AZ (US); Raina M. Maier, Tucson, AZ (US)

(21) Appl. No.: 18/719,497

(22) PCT Filed: Dec. 13, 2022
(86) PCT No.: PCT/US2022/081468

§ 371 Date: Jun. 13, 2024

Related U.S. Application Data
(60) Provisional application No. 63/289,111, filed on Dec. 13, 2021.

(51) Int. Cl. C09K 3/22; E21F 5/06

(52) U.S. Cl. CPC C09K 3/22; E21F 5/06

(57) ABSTRACT

Dust poses a significant threat to human and environmental health. In 2015, an estimated 4.2 million deaths, as well as numerous health issues, were attributed to dust. Unlike other major contaminant transport pathways (e.g., water, soil, biota), atmospheric transmission is relatively unobstructed, able to rapidly transport large masses of materials over long distances and broadly impact areas downwind on local to global scales. Accordingly, dust affects global biogeochemical cycles, pollutes water bodies and air masses, and impacts global climate. Therefore, developing innovative, environmentally-compatible technologies capable of mitigating dust emissions is crucial to protect human and environmental health from mining related dust. The present invention features a method for suppressing dust from a substrate surface, said method comprising applying an effective amount of dust suppressant composition to said substrate surface, wherein said dust suppressant composition comprises an aqueous solution of a glycolipid.

### CROSS-REFERENCES TO RELATED APPLICATIONS

This application claims benefit of U.S. Provisional Application No. 63/289,111 filed Dec. 13, 2021, the specification of which is incorporated herein in their entirety by reference.

### STATEMENT REGARDING FEDERALLY SPONSORED RESEARCH OR DEVELOPMENT

This invention was made with government support under Grant Nos. R43 ES034319 and P42 ES004940 awarded by National Institutes of Health. The government has certain rights in the invention.

### FIELD OF THE INVENTION

The present invention relates to a dust suppressant composition comprising an aqueous solution of glycolipids.

### BACKGROUND OF THE INVENTION

The presence of particulate matter (i.e., dust or fine particles that become suspended in air) is a severe hazard to the environment as well as to the health and safety of individuals. Examples of dust forming materials include iron ores, coal, minerals, and other friable (i.e., a material that is easily broken up into small pieces) particulate matter (PM) ranging from 2 mm to less than 1 μm. Of special interest here are particles of size≤2.5 μm (PM_{2.5}) and ≤10 μm (PM₁₀), which are used to monitor air quality. It is believed that ambient air pollution is one of the leading contributors to the global disease burden that increases morbidity and mortality. In fact, dust has been estimated to account for 4.2 million deaths in 2015. Aside from death, other health impacts of dust are also well documented. As a constituent of total atmospheric particulate matter (PM), dust poses a significant threat to human and environmental health. Unlike other major contaminant transport pathways (e.g., water, soil, biota), atmospheric transmission is relatively unobstructed, able to rapidly transport large masses of materials over long distances and broadly impact areas downwind on local to global scales. Accordingly, dust affects global biogeochemical cycles, pollutes water bodies and air masses, and impacts global climate.

Moreover, dust presents physical (e.g., reduced visibility and explosive mixtures) and health hazards that vary based on characteristics including, but not limited to, particle size distribution, mineral composition, chemical composition, and route of exposure (e.g., respiratory, digestive, skin, membrane, etc.). Harms associated with exposure to dust include damage to the lungs (e.g., silicosis, asbestosis), nose, throat, eyes, and skin (e.g., dermatitis); systematic poisoning; ischemic heart diseases; allergic reactions (e.g., skin rashes, occupational asthma); inflammatory injuries (e.g., chronic bronchitis or emphysema); and various cancers. Indeed, dust storms have significant public health impacts, particularly affecting cardiovascular and respiratory health. Furthermore, health effects can be exacerbated when dust carries contaminants such as organics (e.g., pesticides), metals, metalloids, or biological pathogens (e.g., valley fever).

Mining has disturbed an estimated 57,300 km² globally (0.04% of global land area) and 6,400 km² in the U.S. (0.07% of land area). This is significant because, among anthropogenic dust sources, emissions from mining operations pose one of the greatest potential risks to human health and the environment. At its core, mining is a process of liberating minute amounts of metal from stable, solid matrices. Dust is generated in virtually every step of the mining process: excavating, blasting, stockpiling, crushing, grinding, and transporting. Tailings that remain after ore processing are stored in massive piles subject to wind erosion. Mining in arid and semi-arid regions, such as the Southwestern U.S., is particularly concerning due to characteristically low soil moisture, low atmospheric humidity, and high temperatures that correlate to increased dust emissions. In addition to the mineral particles, dust originating from mines is known to be an exposure route for other contaminants (e.g., arsenic and lead). It is estimated that 60% of all atmospheric arsenic initially originated from mining operation point sources. In Arizona, where mining is highly prevalent, data show that arsenic, lead, copper, or zinc concentrations are elevated on more than half of the days with elevated dust concentrations. Overall, particulate emissions from this industry alone account for 12% of global particulate matter health impacts.

In addition to active mining operations, dust emissions from legacy mining sites are also problematic. There are 22,625 legacy mine features across federal lands that pose environmental hazards (including transport of solid residues) due to the presence of harmful substances, and the U.S. Environmental Protection Agency (EPA) lists 143 mining related sites in the Superfund alternative approach pathway or on the National Priorities List.

Mine tailing particles range in size (diameter) from 2 mm to less than 1 μm. Particle size is a controlling factor in how dust behaves and the risks they pose. In terms of behavior, large particles (>100 μm) settle quickly 6-9 meters from their source, while particles 30-100 μm can travel hundreds of meters, and those <30 μm can travel even greater distances due to slower settling velocities. In terms of inhalation, particles <10 μm penetrate deeply into the respiratory system and tend to be associated with environmental contaminants, thereby posing the most significant health hazard. Due to these risks, the EPA has established 24-hour air quality standards of 12 μg/m³ and 54 μg/m³ for ultrafine (≤2.5 μm; PM_{2.5}) and fine (≤10 μm; PM₁₀) particles, respectively.

Therefore, developing innovative, environmentally-compatible technologies capable of mitigating dust emissions is crucial to protect human and environmental health from mining related dust.

While there are many chemical and mechanical methods for dust suppression, conventional methods have limitations. Mechanical methods of dust suppression include dust collection equipment, which requires expensive equipment. Chemical methods include short- and long-term residual suppressants. Some of the chemical methods include the use of a polymer or binder film over the dusting material. Some chemical methods are relatively expensive, often corrosive to machinery, can cause potential risks to human health due to hazardous components, and have short-term effectiveness due to the fragility of the protection layer that can be easily disrupted by environmental factors, such as strong wind. While water can be used as a dust suppressant, it quickly loses its effectiveness upon evaporation.

Accordingly, there is a continuing need for dust suppression methods that are long-lasting and relatively inexpensive.

### BRIEF SUMMARY OF THE INVENTION

It is an objective of the present invention to provide compositions and methods that allow for the suppression of dust, as specified in the independent claims. Embodiments of the invention are given in the dependent claims. Embodiments of the present invention can be freely combined with each other if they are not mutually exclusive.

In some embodiments, the present invention features a method for suppressing dust from a substrate surface, said method comprising applying an effective amount of dust suppressant composition to said substrate surface, wherein said dust suppressant composition comprises an aqueous solution of a glycolipid.

One of the unique and inventive technical features of the present invention is the use of both biosynthetic and synthetic glycolipids for dust suppression. Without wishing to limit the invention to any theory or mechanism, it is believed that the technical feature of the present invention advantageously provides for a sustainable, environmentally friendly, low toxic, and biodegradable method for suppression. None of the presently known prior references or work has the unique inventive technical feature of the present invention.

Furthermore, the prior references teach away from the present invention. For example, neither biologically-sourced nor bioinspired surfactants, specifically glycolipids, have been previously demonstrated as dust suppression agents. Further, as shown in FIG. 10, control of the glycolipid design allows for the optimization of dust suppression efficacy in ways that are not obvious, e.g., changing sugar head group versus the length and/or number of lipid tails.

Furthermore, the inventive technical features of the present invention contributed to a surprising result. For example, it was found that rhamnolipid glycolipids have high efficacy in suppressing dust formation. Rhamnolipids are microbially-produced surfactants that exhibit high surface activity (e.g., reduce surface and interfacial tensions), and are considered green molecules due to their natural production by bacteria and their biodegradability and low toxicity. However, biosynthesized rhamnolipids are produced as ill-defined and inconsistent mixtures making quality control a challenge when developing applications for these materials. The advantage of synthetic glycolipids is not only quality control, but more importantly, the ability to tune the glycolipid structure for particular applications. In particular, synthetic glycolipids can be tailor made depending on desired applications. For example, one can select different sugar moiety (1 or more sugars), the glycosyl linkage (e.g., —O—) can be modified (e.g., —O— can be replaced with —S—, or —NRa—, where Ra is H, C1-10 alkyl, or a nitrogen protecting group), and the fatty acid moiety (including length, number, saturation) of the lipid portion can also be modified.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings of various glycolipids in which:

FIG. 1A shows the structure of a biosynthetic Rhamnolipid where ‘n’ and ‘m’ vary from 2 to 20 creating a complex mixture of congeners.

FIG. 1B shows a synthetic Xylolipid-C10C10 glycolipid that exhibits the three tailorable structure moieties that can be achieved synthetically: hydrophilic sugar, linkage, and hydrophobic tail(s).

FIG. 1C shows the structure of a biosynthetic Dirhamnolipid mixture where ‘n’ and ‘m’ vary from 2 to 20 creating a complex mixture of congeners.

FIG. 1D shows a single synthetic Dirhamnolipid-C10C10 congener.

FIGS. 1E and 1F show examples for formula II and an alkyl, respectively.

FIG. 2A shows representative glycolipids tested as hydrophilic dust (e.g., mine tailings) suppressants.

FIG. 2B shows an expanded suite of representative glycolipids tested as hydrophilic dust (e.g., mine tailings) suppressants.

FIG. 3 shows representative glycolipids tested as hydrophilic and hydrophobic dust (e.g., mine tailings or coal dust, respectively) suppressants

FIG. 4 shows a schematic illustration of wind erosion testing apparatus. Various compressed air pressure can be applied to the sample surface. The dust sensor records PM_{2.5} and PM₁₀ (μg/m³) in real time. After the measurement, a wind speed sensor is located at the sample surface to convert the air pressure to wind speed.

FIG. 5 shows results of wind erosion test on dust samples from mine tailings. Sample beds were treated by 1 w/v % of Rhamnolipid-C10 (Rha C10), -C14 (Rha C14) and -C18 (Rha C18) in water. PM₁₀ and PM_{2.5} concentrations in air were measured after the water evaporation. Wind speed on the sample surface: 55 km/h.

FIG. 6 shows results of wind erosion test on dust samples from mine tailings. Sample beds were treated by 1 w/v % of Rhamnolipid-C10C10 (Rha C10C10), -C12C12 (Rha C1212), -C14C14 (Rha C14C14), and Dirhamnolipid-C10C10 (Dirha C10C10) in water. Wind speed on the sample surface: 55 km/h.

FIG. 7 shows results of wind erosion test on dust samples from mine tailings. Sample beds were treated by 1 w/v % of Xylolipid-C10 (Xyl C10), -C14 (Xyl C14), -C18 (Xyl C18), -C10C10 (Xyl C10C10) and -C14C14 (Xyl C14C14) in water. After water evaporation, 55 km/h wind applied to the sample to measure PM₁₀ and PM_{2.5}.

FIG. 8 shows results of wind erosion test on dust samples from mine tailings. Sample beds were treated by 1 w/v % of 1-oxy-melibioside C8 (Ls255M-shown), 1-oxy-melibioside C10 (Ls249M) and 1-oxy-melibioside C12 (Ls253M) in water. After water evaporation, 55 km/h wind applied to the sample to measure PM10 and PM_{2.5}.

FIGS. 9A and 9B show results of wind erosion tests on dust samples from mine tailings. Sample beds were treated by (FIG. 9A) 1 w/v % of 1-oxy-cellobioside C8 (Ls263M-shown), 1-oxy-cellobioside C10 (Ls261M) and 1-oxy-cellobioside C12 (Ls265M) in water; and (FIG. 9B) 1 w/v % of 2-oxy-cellobioside C8 (Ls286-shown), 2-oxy-cellobioside C10 (Ls268M) and 2-oxy-cellobioside C12 (Ls285) in water. After water evaporation, 55 km/h wind applied to the sample to measure PM₁₀ and PM_{2.5}

FIG. 10 shows a summary table of wind erosion test against tailings dust treated with glycolipids. ¹Note: EPA AQI categories are based on the results of PM_{2.5}, more hazardous to human health than PM₁₀.

FIG. 11 shows captured images from videos that recorded the sink test. Coal particles (<100 μm in diameter; total 0.5 g) were dropped onto aqueous solutions, containing 0.1 w/v % of Octyl β-D-glucopyranoside (Octyl), biosynthetic Dirhamnolipid and Rhamnolipid C12C12 (Rha C12C12).

FIGS. 12A and 12B show results of analysis of settling coal particles in solution, containing 0.1 w/v % glycolipids in the presence (FIG. 12A) and absence (FIG. 12B) of 0.1 w/v % HPMC. Coal particles (<100 μm in diameter; total 0.5 g) were dropped on top of water and their wetting was video-recorded. Sizes and numbers of settling particles were analyzed by using Image J software.

FIG. 13 shows snapshots of settling coal particles in solution, containing 0.1 w/v % glycolipids in the presence and absence of 0.1 w/v % HPMC. Coal particles (<100 μm in diameter; total 0.5 g) were dropped on top of water and their wetting was video-recorded.

FIG. 14 shows results of wind erosion test on dust samples from mine tailings. Sample beds were treated by 1 w/v % of Xyloside-C10 (XyIE C10), -C12 (XyIE C12), and -C14 (XyIE C14) in water. After water evaporation, 55 km/h wind applied to the sample to measure PM₁₀ and PM_{2.5}.

FIG. 15 shows results of wind erosion test on dust samples from mine tailings. Sample beds were treated by 1 w/v % of Galactolipid-C10 (Gal C10) and -C14 (Gal C14) in water. After water evaporation, 55 km/h wind applied to the sample to measure PM₁₀ and PM_{2.5}.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

When referring to glycolipids of the invention, the term “derivative” refers to any chemical modification of the parent compound or a compound derived from the parent compound. For example, a derivative of a carbohydrate includes an alkylated carbohydrate, replacement of one or more hydroxyl groups with hydrogen, halide, amine, or a thiol; modification of a hydroxyl group (e.g., by esterification, etherification, protection, etc.); as well as other derivatives known to one skilled in the art. The term carbohydrate includes pyranose and furanose carbohydrates. Exemplary derivatives of carbohydrates include, but are not limited to, alkylated or carboxylated carbohydrates (e.g., one or more hydroxyl groups that are methylated, ethylated, acetylated, or benzoylated), thiol carbohydrate (where one or more hydroxyl groups are replaced with —SH moiety), deoxy carbohydrates (where one or more-OH groups of the carbohydrate are replaced with —H), amine carbohydrates (where one or more-OH groups of the carbohydrate are replaced with —NR^{a}R^{b}, where each of R^{a} and R^{b} is independently H, C1-C6 alkyl, or a nitrogen protecting group, etc. More specifically, when referring to a carbohydrate, the term “derivative thereof” refers to a derivative of a carbohydrate in which one or more of the hydroxyl groups is replaced with hydrogen (e.g., 2-deoxy glucose, 5-deoxyglucose, etc.), an amine (e.g., amino sugars), a thiol (—SH) or a halogen, such as chloro, fluoro or iodo, (e.g., 5-fluoroglucose, 2-fluoroglucose, 5-chrologlucose, 2-chloroglucose, etc.). In addition, each of the monosaccharides can be an (L)-isomer or a (D)-isomer. The term “a thiol derivative” of a sugar refers to a sugar moiety in which the hydroxyl group that links the “B” moiety in a compound according to Formula I is replaced with a sulfur atom (e.g., the linkage between A and B moieties in a compound according to Formula I is sulfur). Similarly, the term “an amine or amino derivative” of a sugar refers to a sugar moiety in which the hydroxyl group that links the “B” moiety in the compound according to Formula I is replaced with a nitrogen atom (e.g., the linkage between A and B is achieved by —NH— moiety).

The term “sugar” and “carbohydrate” are used interchangeably herein and generally refers to a mono- or disaccharide or mixtures thereof. Exemplary carbohydrates that can be used in methods of the invention include, but are not limited to, the following carbohydrates:

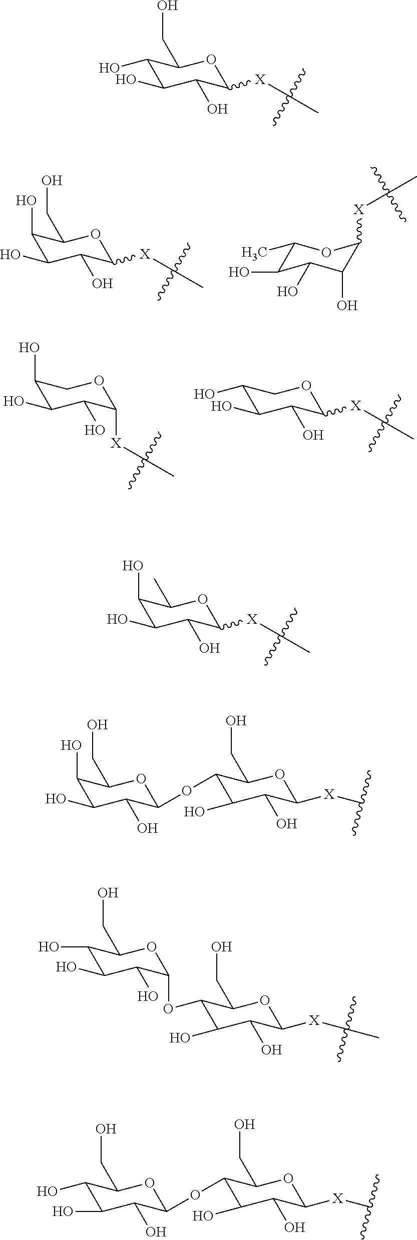

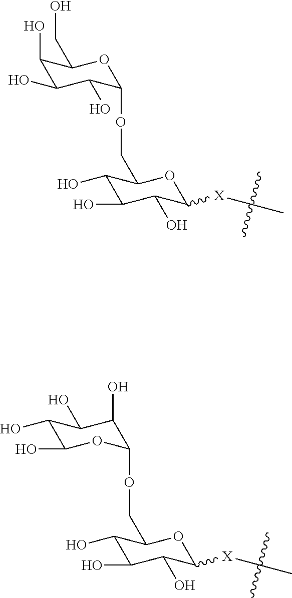

where X is O or S, and where one or more-OH is replaced with H, halogen, or —OR, where R is C18 alkyl.

The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₈H₁₂O₆ or a derivative thereof. Exemplary monosaccharides of the invention include but are not limited to, allose, altrose, arabinose, fructose, galactose, glucose, gulose, idose, lxyose, psicose, rhamnose, ribose, ribulose, sorbose, tagatose, talose, xylose, xylulose, and derivative thereof. Each monosaccharide can also be independently an (L)-isomer or a (D)-isomer.

The term “disaccharide” refers to a carbohydrate composed of two monosaccharides. It is formed when two monosaccharides are covalently linked to form a dimer. The linkage can be a (1-4) bond, a (1-6) bond, a (1-2) bond, a (1-3) bond, etc. between the two monosaccharides. In addition, each of the monosaccharides can be independently an α- or β-anomer. Exemplary disaccharides that can be used in the present invention include, but are not limited to, cellobiose, chitobiose, dirhamnose, gentiobiose, isomaltose, isomaltulose, lactose, lactulose, laminaribose, leucrose, maltose, maltulose, melibiose, nigerose, sophorose, sucrose, terhalose, turanose, xylobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Each monosaccharide can also be independently an (L)-isomer or a (D)-isomer.

As used herein, the term “dust” may refer to a fine, dry powder comprising small particles of solid matter (e.g., earth or waste matter) lying on the ground or on surfaces or carried in the air. In some embodiments, dust forming materials include iron ores, coal, minerals, and other friable (i.e., a material that is easily broken up into small pieces) materials. Examples of dust forming materials include iron ores, coal, minerals, and other friable (i.e., a material that is easily broken up into small pieces) with particulate matter (PM) ranging from 2 mm to less than 1 μm. Of special interest here are particles of size≤2.5 μm (PM_{2.5}) and ≤10 μm (PM₁₀) which are used to monitor air quality. It is

As used herein, ‘conventional surfactants’ may refer to conventional petroleum-based surfactants (e.g., sodium dodecyl sulfate) that may have deleterious toxicological and environmental issues. Thus, instead of using potentially toxic, conventional surfactants, the present invention utilizes eco-friendly bioinspired glycolipids that are renewably-sourced, sustainable, environmentally friendly, low toxicity, and biodegradable for dust suppression.

As used herein, the term “substrate” may refer to a solid substance or medium to which compositions described herein may be applied

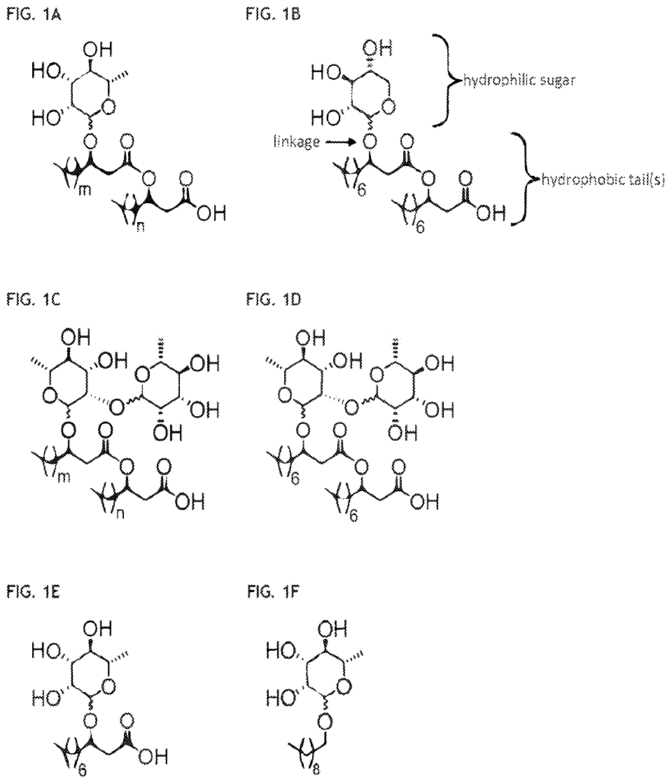

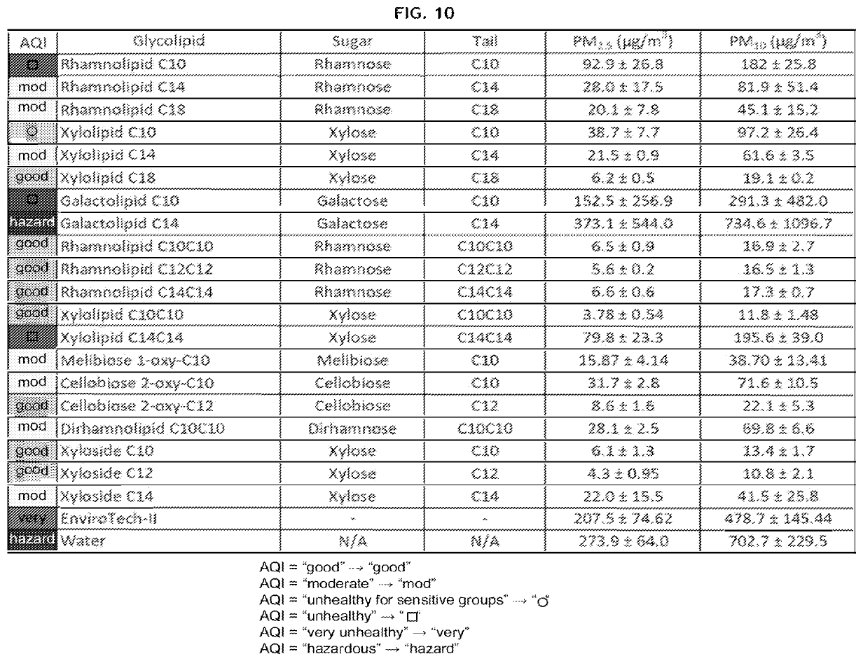

Referring now to FIGS. 1A-15, the present invention features a method for suppressing dust from a substrate surface. The method may comprise applying an effective amount of dust suppressant composition to the said substrate surface. Example ideal compositions could contain, but are not limited to, any glycolipid in FIG. 10 that produces a “good” AQI rating, such as Xyloside-C10, Xyloside-C12, Xylolipid-C18, Rhamnolipid-C10C10, Rhamnolipid-C14C14, or Xylolipid-C10C10. In some embodiments, the dust suppressant composition comprises an aqueous solution of a glycolipid. In some embodiments, the present invention features a method for suppressing airborne dust. The method may comprise applying an effective amount of dust suppressant composition to an area, including, but not limited to, mining areas (tailings, haul roads, material transfer systems, waste rock storage, grinding and milling activities), agriculture (fallow or active fields), dirt roads, fine material handling processes, and enclosed spaces with dust generating activities.

Non-limiting examples of a substrate may include but are not limited to mine tailings, waste rock, soil, coal, a coal mine, fly ash, dust, or a combination thereof. In some embodiments, the substrate may be dust (e.g., coal dust) or fly ash. For example, water droplets, containing the compositions described herein, may be sprayed into the air such as to bind to dust in the air. Without wishing to limit the present invention to any theory or mechanism, it is believed that when water droplets, containing the compositions described herein, are sprayed into the air, the compositions enhance binding of the dust (e.g., the surface of the dust) to the water droplets and increase the total weight of the mixture; this, in turn, allows the dust to settle faster and helps to control airborne dust.

In some embodiments, the glycolipid comprises the formula:

A-[B]ₐ (Formula I)

wherein a is 1 or 2, and wherein A is selected from the group consisting of a monosaccharide, a disaccharide, and a derivative thereof; and each B is attached to a different position of A and is independently a C₆-C₂₀ alkyl or a moiety of the formula:

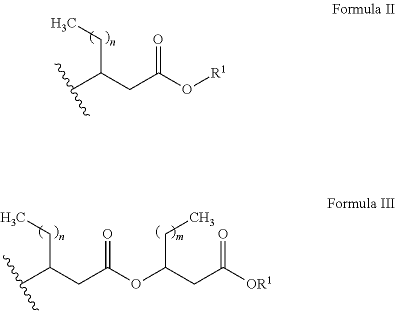

wherein each of m and n is independently an integer from 2 to about 20, and R¹ is H or C₁-C₂₀ alkyl.

The above-mentioned Formula II has one chiral center, whereas Formula III has two chiral centers. While not necessary, one can use enantiomerically enriched moieties of Formula II or Formula III. In general, for cost considerations, a racemic mixture of Formula II or Formula III may be used.

In some embodiments, a is 1. In other embodiments, a is 2. In some embodiments, if a is 1, B (i.e., the fatty acid or lipid moiety) is attached to the anomeric carbon on A (i.e., the sugar moiety). Alternatively, if a is 2, B (i.e., the fatty acid or lipid moiety) may be attached at multiple sites on A (i.e., the sugar moiety), typically a hydroxyl on A or a thiol/amine derivative of A.

In some embodiments, A is a sugar comprising a monosaccharide or a disaccharide. In some embodiments, A is a monosaccharide or a thiol derivative thereof, or an amine derivative thereof. Non-limiting examples of monosaccharides may include but are not limited to glucose, galactose, rhamnose, arabinose, xylose, fructose, or fucose.

In some embodiments, B is attached to the hydroxyl group of the anomeric carbon or a thiol derivative thereof or an amine derivative thereof of said monosaccharide.

In other embodiments, A is a disaccharide, or a thiol derivative thereof or an amine derivative thereof. The disaccharide used herein may comprise a 1-2, 1-4, or 1-6 linkage between two monosaccharides. Non-limiting examples of disaccharides may include but are not limited to lactose, maltose, sucrose, melibiose, cellobiose, or rutinose.

Yet still, in other embodiments, each of m and n is independently an integer from 2 to 20.

In one particular embodiment, B is a moiety of the formula:

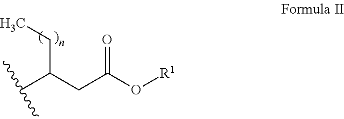

wherein n and R¹ are those defined herein. As used herein, the terms “those defined above” and “those defined herein” when referring to a variable incorporate by reference the broad definition of the variable as well as any narrower definition(s), if any.

In another particular embodiment, B is a moiety of the formula:

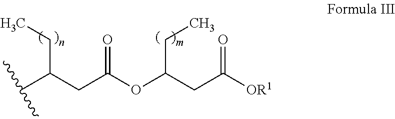

wherein m, n, and R¹ are those defined herein.

In some embodiments, R¹ comprises a hydrogen (H). In other embodiments, R¹ comprises an alkyl group (e.g., a straight-chain alkyl group). For example, R¹ may comprise an alkyl group comprising 1 to 20 carbons. In some embodiments, R¹ may comprise an alkyl group comprising about 1 to 20 carbons, or about 1 to 15 carbons, or about 1 to 10 carbons, or about 1 to 5 carbons, or about 5 to 20 carbons, or about 5 to 15 carbons, or about 5 to 10 carbons, or about 10 to 20 carbons, or about 10 to 15 carbons, or about 15 to 20 carbons.

In some embodiments, R¹ is a straight-chain alkyl group; and not a branch alkyl group. Without wishing to limit the present invention to any theory or mechanism, it is believed that a branch alkyl group may increase toxicity and limit biodegradability.

In some embodiments, n is an integer from 1 to 30, or 1 to 25, or 1 to 20, or 1 to 15, or 1 to 10, or 1 to 6, or 1 to 5, or 1 to 2, or 2 to 30, or 2 to 25, or 2 to 20, or 2 to 15, or 2 to 10, or 2 to 6, or 2 to 5, or 5 to 30, or 5 to 25, or 5 to 20, or 5 to 15, or 5 to 10, or 5 to 6, or 6 to 30, or 6 to 25, or 6 to 20, or 6 to 15, or 6 to 10, or 10 to 30, or 10 to 25, or 10 to 20, or 10 to 15, or 15 to 30, or 15 to 25, or 15 to 20, or 20 to 30, or 20 to 25, or 25 to 30. Typically n is an integer from 2 to about 25, often from 3 to about 25, and most often from 6 to about 20. Still, in other particular embodiments, n is independently 2, 4, 6, 8, 10, 12, 14, 16, 18, or 20.

In some embodiments, m is an integer from 1 to 30, or 1 to 25, or 1 to 20, or 1 to 15, or 1 to 10, or 1 to 6, or 1 to 5, or 1 to 2, or 2 to 30, or 2 to 25, or 2 to 20, or 2 to 15, or 2 to 10, or 2 to 6, or 2 to 5, or 5 to 30, or 5 to 25, or 5 to 20, or 5 to 15, or 5 to 10, or 5 to 6, or 6 to 30, or 6 to 25, or 6 to 20, or 6 to 15, or 6 to 10, or 10 to 30, or 10 to 25, or 10 to 20, or 10 to 15, or 15 to 30, or 15 to 25, or 15 to 20, or 20 to 30, or 20 to 25, or 25 to 30. Typically m is an integer from 2 to about 25, often from 3 to about 25, and most often from 6 to about 20. Still, in other particular embodiments, m is independently 2, 4, 6, 8, 10, 12, 14, 16, 18, or 20.

Typically, the aqueous solution used as a dust suppressant comprises 1% w/v or less of the glycolipids described herein. In some embodiments, the aqueous solution comprise about 0.001% w/v to 10% w/v, or about 0.001% w/v to 8% w/v, or about 0.001% to 5% w/v, or about 0.001% to 4% w/v, or about 0.001% w/v to 3% w/v, or about 0.001% w/v to 2% w/v, or about 0.001% w/v to 1% w/v, or about 0.001% w/v to 0.1% w/v, or about 0.001% w/v to 0.01% w/v, or about 0.01% w/v to 10% w/v, or about 0.01% w/v to 8% w/v, or about 0.01% to 5% w/v, or about 0.01% to 4% w/v, or about 0.01% w/v to 3% w/V, or about 0.01% w/v to 2% w/v, or about 0.01% w/v to 1% w/v, or about 0.01% w/v to 0.1% w/v, or about 0.1% w/v to 10% w/v, or about 0.1% w/v to 8% w/v, or about 0.1% to 5% w/v, or about 0.1% to 4% w/v, or about 0.1% w/v to 3% w/v, or about 0.1% w/v to 2% w/v, or about 0.1% w/v to 1% w/v, or about 1% w/v to 10% w/v, or about 1% w/v to 8% w/v, or about 1% to 5% w/v, or about 1% to 4% w/v, or about 1% w/v to 3% w/v, or about 1% w/v to 2% w/v, or about 2% w/v to 10% w/v, or about 2% w/v to 8% w/v, or about 2% to 5% w/v, or about 2% to 4% w/v, or about 2% w/v to 3% w/v, or about 3% w/v to 10% w/v, or about 3% w/v to 8% w/v, or about 3% to 5% w/v, or about 3% to 4% w/v, or about 4% w/v to 10% w/v, or about 4% w/v to 8% w/v, or about 4% to 5% w/v, or about 5% w/v to 10% w/v, or about 5% w/v to 8% w/v, or about 8% w/v to 10% w/v of the gylcolipids described herein. In some embodiments, the aqueous solution used as dust suppressant comprises about 0.001% w/v, or about 0.001% w/v, or about 0.01% w/v, or about 0.1% w/v, or about 1% w/v, or about 2% w/v, or about 3% w/v, or about 4% w/v, or about 5% w/v, or about 6% w/v, or about 7% w/v, or about 8% w/v, or about 9% w/v, or about 10% w/v of the glycolipids described herein.

In some embodiments, the dust suppressant composition described herein (e.g., an aqueous solution comprising a glycolipid) may comprise a pH of about 5 to 9. In other embodiments, the dust suppressant composition described herein may comprise a pH of about 4 to 10, or about 4 to 9, or about 4 to 8, or about 4 to 7, or about 4 to 6, or about 4 to 5, or about 5 to 10, or about 5 to 9, or about 5 to 8, or about 5 to 7, or about 5 to 6, or about 6 to 10, or about 6 to 9, or about 6 to 8, or about 6 to 7, or about 7 to 10, or about 7 to 9, or about 7 to 8, or about 8 to 10, or about 8 to 9, or about 9 to 10.

The amount of dust suppressant solution used can vary depending on a wide variety of factors including, but not limited to, the surface to be treated, ambient temperature, ambient humidity, etc. In one particular embodiment, the composition (i.e., dust suppressant aqueous solution of a glycolipid) is applied at a rate of at least about 0.25 L/m², typically at least about 0.5 L/m², often at least about 1 L/m², and most often at least about 2 L/m². Alternatively, from about 50,000 to about 600,000 gallons per km², typically from about 60,000 to about 400,000 gallons per km², often from about 100,000 to about 300,000 gallons per km², and most often from about 150,000 to about 250,000 gallons per km².

In some embodiments, the dust suppressant composition described herein (e.g., an aqueous solution of a glycolipid) may further comprise a crust forming enhancer that assists in the aggregation of particles. In some embodiments, the dust suppressant composition described herein (e.g., an aqueous solution of a glycolipid) may further comprise cellulose. In some embodiments, the dust suppressant composition described herein (e.g., an aqueous solution of a glycolipid) may further comprise alkylated cellulose. In one particular embodiment, the alkylated cellulose compound is hydroxypropyl methyl cellulose (HPMC). Non-limiting examples of alkylated cellulose include but are not limited to methyl cellulose or ethyl cellulose. In certain embodiments, the dust suppressant composition described herein may further comprise hydroxypropyl methyl cellulose (HPMC).

Without wishing to limit the present invention to any theory or mechanism, it is believed that the dust suppressant compositions described herein (e.g., an aqueous solution of a glycolipid) effectively suppress hydrophobic dust by synergistically wetting and aggregating the dust particles, which is important for health protection as larger particles pose fewer risks, are more easily removed by sedimentation, and less prone to being re-deposited in air.

Another aspect of the invention provides a method for reducing formation of dust from a substrate surface (e.g., soil). The method includes applying a dust suppressant composition to said substrate surface (e.g., soil), wherein said dust suppressant composition comprises an aqueous solution of a glycolipid comprising the formula:

A-[B]ₐ (Formula I)

wherein a is 1 or 2; A is selected from the group consisting of a monosaccharide, a disaccharide, and a derivative thereof; and each B is attached to a different position of A and is independently a C6-C20 alkyl or a moiety of the formula:

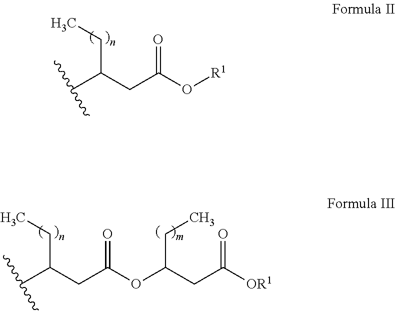

wherein each of m and n is independently an integer from 2 to about 20; and R¹ is H or C1-C20 alkyl.

Glycolipids of the present invention can be readily prepared using, for example, procedures disclosed in commonly assigned U.S. patent application Ser. No. 15/358,159, which is incorporated herein by reference in its entirety.

### Example

The following is a non-limiting example of the present invention. It is to be understood that said example is not intended to limit the present invention in any way. Equivalents or substitutes are within the scope of the present invention.

### Materials and Methods

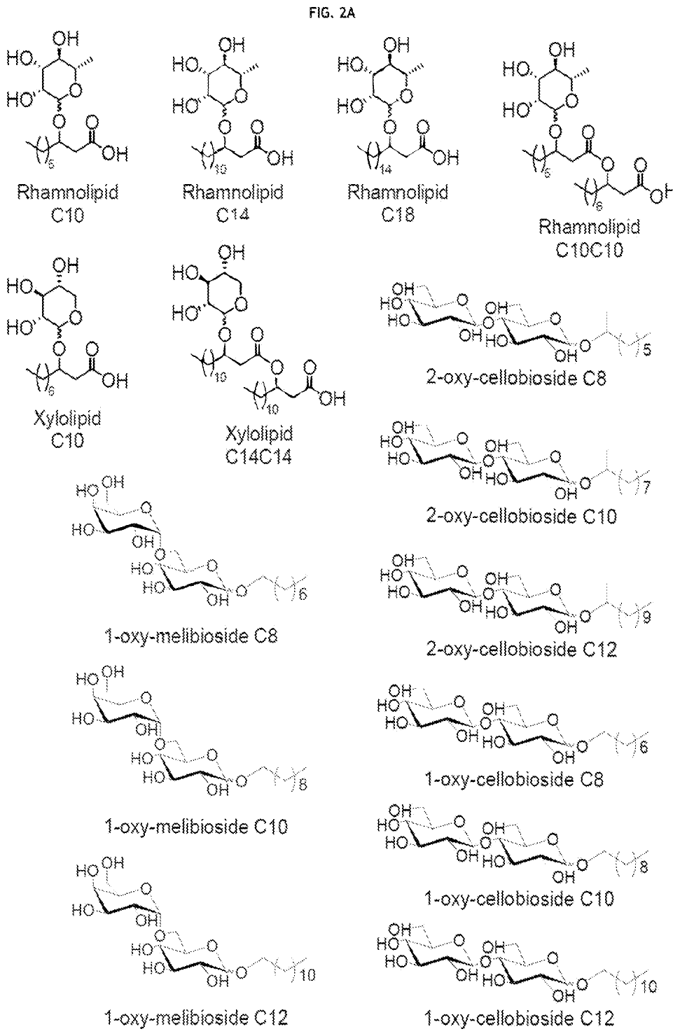

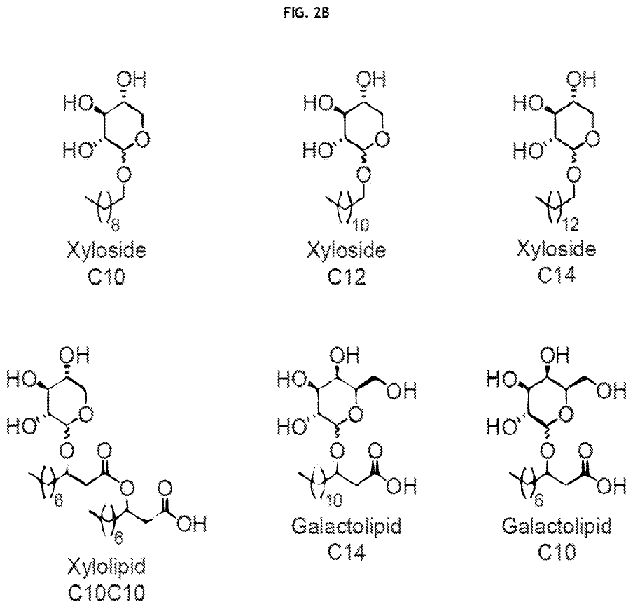

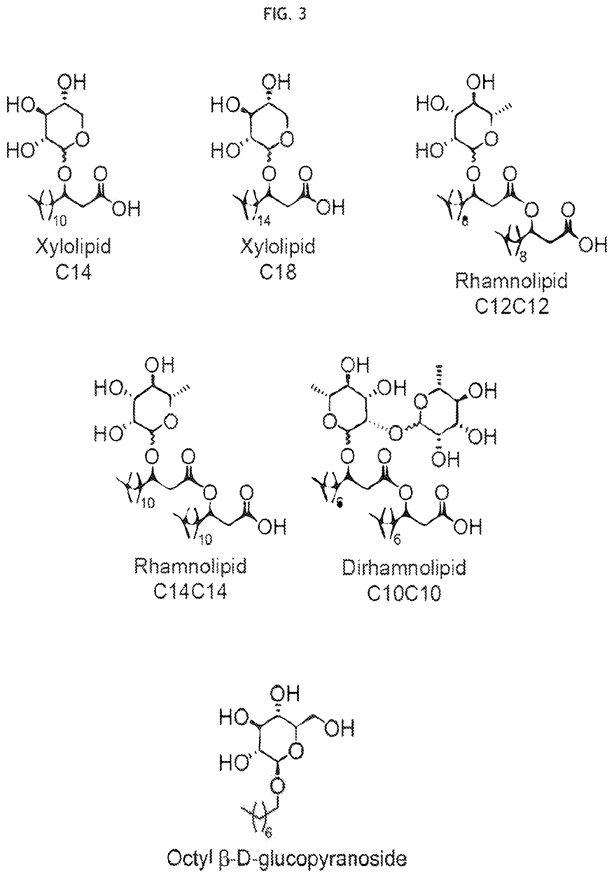

Glycolipids: The following synthetic glycolipids were tested for suppression of dust from mine tailings: Rhamnolipid C10, C14, C18, C10C10, C12C12 and C14C14; Dirhamnolipid C10C10; Xylolipid C10, C14, C18, C10C10, and C14C14; Xyloside C10, C12, C14; Galactolipid C10, C14; 1-oxy-melibioside C8, C10, C12; 1-oxy-cellobioside C8, C10, C12; and 2-oxy-cellobioside C8, C10, C12. In addition a biosynthetic Dirhamnolipid which contains a mixture of congeners, was tested. See FIGS. 2A and 2B, and 3.

The following synthetic glycolipids were tested for their ability to wet coal dust: Octyl β-D-glucopyranoside (Octyl); Xylolipid C10, C14, C18; Rhamnolipid C10C10, C12C12, C14C14; Rhamnolipid C10, C14, C18; Gal C18; 1-oxy-melibioside C10, C12; 1-oxy-cellobioside C8, C10, C12; 2-oxy-cellobioside C8, C10, C12; thio-glucoside C10; thio-galactoside C10; thio-cellobioside C10; thio-maltibioside C10; and thio-lactoside C10. In addition, a biosynthetic Dirhamnolipid which contains a mixture of congeners, was tested. See FIGS. 2A and 2B, and 3.

Cellulose: Cellulose is an attractive dust controlling material due to its sustainability and biodegradability. Active hydroxyl groups (—OH) on the D-glucopyranose units of celluloses enable intramolecular and intermolecular hydrogen bonding, making cellulose useful as a binder, thickener, emulsifier, and dispersing agent in food, cosmetics, and pharmaceuticals. Substituting the hydroxyl groups of cellulose with methyl groups (—CH3) produces methyl celluloses (MCs) with increased amphiphilicity. Among MCs, hydroxypropyl methyl cellulose (HPMC; 100,000 cps or mPas at 25° C. in 2 w/v %) of 0.1 w/v % was used together with various biosurfactants during the wettability test of coal particles.

Dust sample preparation: Samples of mine tailings and coal granules were dried in the electric oven at 50° C. for a week to remove any residual moisture. Dried coal granules were crushed and ground by mortar and pestle, then sieved with a US standard 140 mesh (opening size: <100 μm).

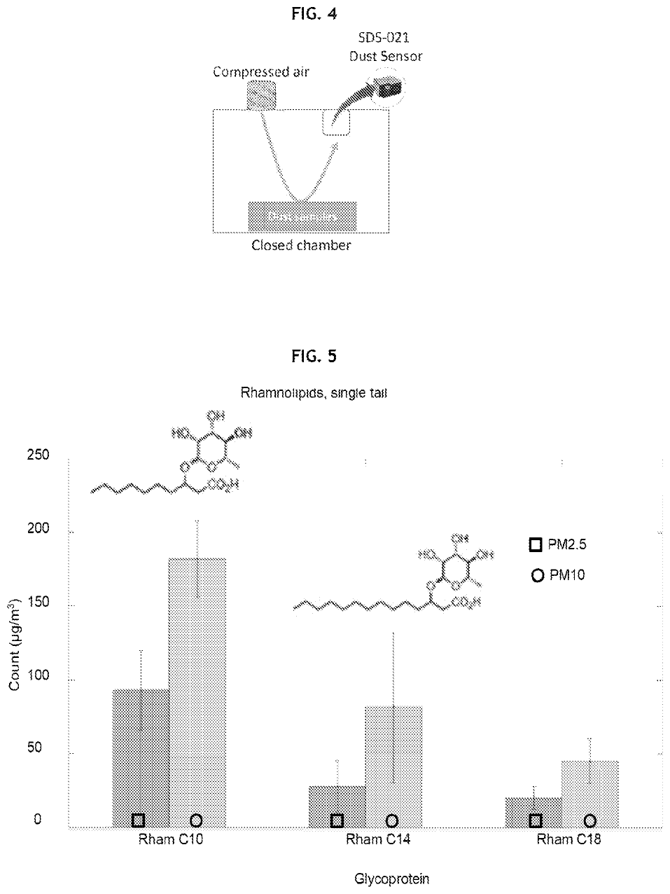

Wind erosion test. Sample beds were prepared by weighing 1.5 g of mine tailing particles on a weighing boat (501215168, Fisher Scientific, MA, USA) and then spraying the material with 2 mL of the test solutions containing glycolipids or only water on the sample surface. The sample beds were dried in a fume hood at room temperature for a week. The sample beds were placed inside a homemade wind erosion testing apparatus (FIG. 4) and then exposed to wind conditions. The test chamber was equipped with a laser dust sensor (SDS-021, Nova Fitness, China) connected to a Raspberry Pi computer module (Raspberry Pi 3 Model B, Vilros, NJ, USA) for data acquisition. PM₁₀ and PM_{2.5} concentrations were recorded every second for 1 min after applying 15 m/s airflows to the sample surface for 10 sec. The PM sensor can detect PM₁₀ and PM_{2.5} at concentrations up to 2,000 μg/m³ and 1,000 μg/m³ with ±15% accuracy, respectively.

Wettability test of coal particles: Coal particle wettability was evaluated by performing a sink test in each of the aqueous solutions containing only biosurfactants or the mixture of biosurfactants and 0.1 w/v % HPMC. Glass vials (Shell Vial, catalog #60965D, Kimble®, USA) were filled with 5 mL of each solution, and 0.5 g of prepared coal particles were dropped on top of the solutions and video-recorded. Snapshots from the video record were taken after one minute and processed to calculate the average sizes of the settling particles and the quantity of total counted particles using ImageJ software.

### Wind Erosion Test Against Mine Tailings Dust:

EPA Air Quality Index (AQI): During the wind erosion test, PM₁₀ and PM_{2.5} were measured, and the possibility of glycolipids as dust suppressants was determined by AQI category as follows:

| | | | Unhealthy for | | Very | |
|---|---|---|---|---|---|---|
| | Good | Moderate | sensitive groups | Unhealthy | unhealthy | Hazardous |
| PM₁₀ (μg/m³) | 0.0-54 | 55-154 | 155-254 | 255-354 | 355-424 | >424 |
| PM_{2.5} (μg/m³) | 0.0-12 | 12.1-35.4 | 35.5-55.4 | 55.5-150.4 | 150.5-250.4 | >250.5 |

### Rhamnolipid—Single Tail:

Against 55 km/h wind speed, 1 w/v % of Rhamnolipid C18 which has the longest single tail suppressed dust particles better than other shorter single tails, and reached to AQI “Good” level for PM₁₀ (FIG. 5). PM_{2.5} was slightly higher than 12.1 μg/m³ and reached the AQI “Moderate” level when using Rhamnolipid C18, which still suppressed dust better than other Rhamnolipid with shorter tails. Based on obtained data, it can be concluded that a rhamnose sugar head with a single tail of C18 or longer is necessary for effective dust suppression to reach the AQI “Good” level.

| | Rhamnolipid | Rhamnolipid | Rhamnolipid |
|---|---|---|---|
| | C18 | C14 | C10 |
| PM₁₀ (μg/m³) | Good | Moderate | Unhealthy for |
| | | | sensitive groups |
| PM_{2.5} (μg/m³) | Moderate | Moderate | Unhealthy |

### Rhamnolipid—Single Tail—Double Tail

All tested single-head Rhamnolipid with double tails significantly suppressed tailings dust against the 55 km/h wind and reached the AQI “Good” level (FIG. 6). The double sugar head rhamnolipid did not suppress the tailings as efficiently as the single head rhamnolipids with double tails (FIG. 6). When comparing Rhamnolipid C18 and Rhamnolipid C10C10 which have similar total tail length, Rhamnolipid C10C10 reduced PM₁₀ and PM_{2.5} 2-3 times more. Therefore, it can be concluded that a double-tailed, single rhamnose sugar head is a more effective dust suppressant (FIG. 6) than the single-tailed one (FIG. 5).

| | Rhamnolipid | Rhamnolipid | Rhamnolipid | Dirhamnlipid |
|---|---|---|---|---|
| | C14C14 | C12C1 | C10C10 | C10C10 |
| PM₁₀ (μg/m³) | Good | Good | Good | Moderate |
| PM_{2.5} (μg/m³) | Good | Good | Good | Moderate |

### Xylolipid-Single and Double Tails

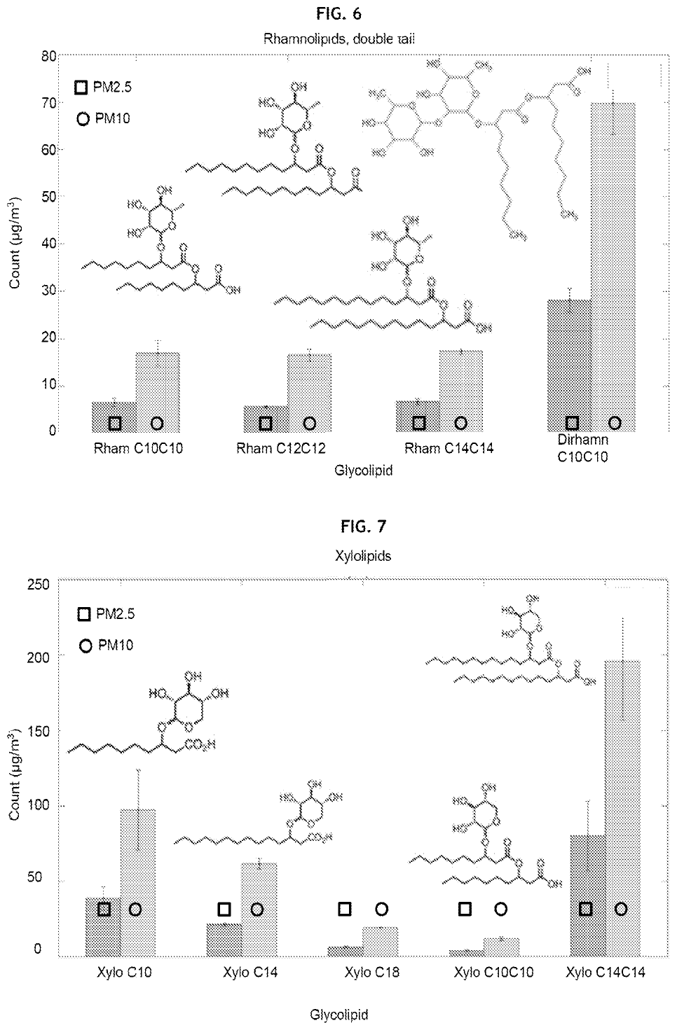

Xylolipid C18 which has the longest single tail suppressed tailings dust greater than other tested Xylolipids with shorter single tails and reached to the AQI “Good” level (FIG. 7). The shortest double tail suppressed the most tailings of all the xylolipids while the longer chain length actually gave worse dust suppression (Xylolipid C10C10 vs. Xylolipid C14C14). Compared to the dust suppression results from Rhamnose sugar head with single or double tails (FIGS. 5 and 6), xylose sugar head with the double tail did improve dust suppression but only to a certain chain length, when the chain length was greater than C10C10, xylose sugar head with double tails worsened the dust suppression, compared to the single tails (FIG. 7). Therefore, it can be concluded that xylose sugar heads with only single tails, specifically C18 or longer, can be considered as effective dust suppressants as well as double tails that are close to C10C10 in length.

| | Xylolipid | Xylolipid | Xylolipid | Xylolipid | Xylolipid |
|---|---|---|---|---|---|
| | C18 | C14 | C10 | C10C10 | C14C14 |
| PM₁₀ (μg/m³) | Good | Moderate | Moderate | Good | Unhealthy for |
| | | | | | sensitive groups |
| PM_{2.5} (μg/m³) | Good | Moderate | Unhealthy for | Good | Unhealthy |
| | | | sensitive groups | | |

### Xyloside-Single Tail

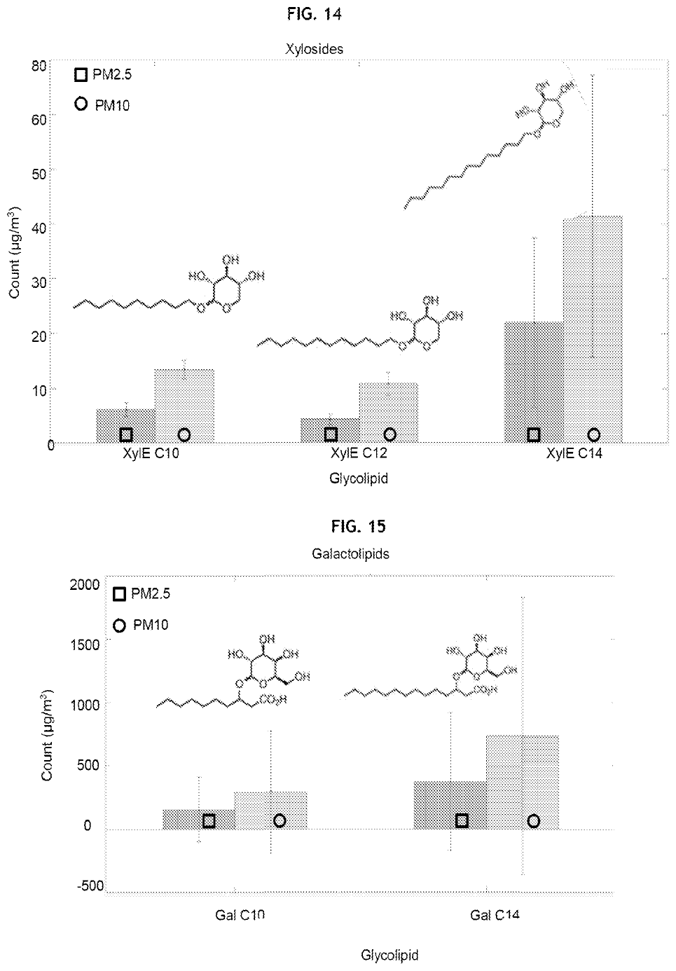

The nonionic, xylose sugar head with a single tail improved dust suppression up to a certain point. Tail lengths of C10 and C12 gave good air quality while the length of C14 gave moderate air quality (FIG. 14). This is similar to the behavior of the xylose sugar head, double tail dust suppression, where a tail length of C10C10 gave good air quality while the length of C14C14 gave unhealthy air quality (based off the PM_{2.5} concentration) (FIG. 7). It can be concluded that nonionic, xylose sugar heads with a single tail of length C10 or C12 are effective in dust suppression.

| | Xyloside C10 | Xyloside C12 | Xyloside C14 |
|---|---|---|---|
| PM₁₀ (μg/m³) | Good | Good | Moderate |
| PM_{2.5} (μg/m³) | Good | Good | Moderate |

### Galactolipid-Single Tail

The galactose sugar head single tail gave very poor air quality, with tail length of C10 giving very unhealthy air quality (based off PM_{2.5} concentration) while C14 gave hazardous air quality (FIG. 15). Based on these results, it can be concluded that galactolipids are useful in effectively suppressing dust.

| | Galatolipid C10 | Galactolipid C14 |
|---|---|---|
| PM₁₀ (μg/m³) | Unhealthy | Hazardous |
| PM_{2.5} (μg/m³) | Very Unhealthy | Hazardous |

### Melibioside: 1-Oxy-Melibioside—Single Tail

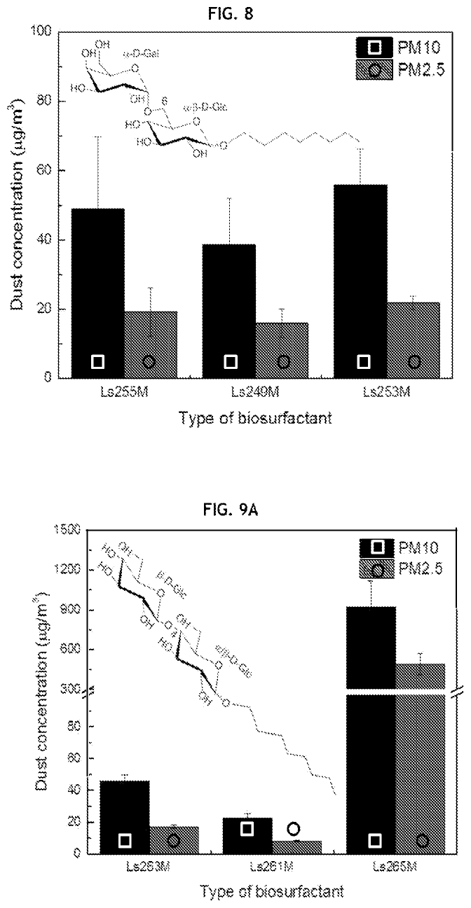

1-Oxy melibioside with single tails of C8, C10 and C12 showed similar dust suppressing capability (FIG. 8). While PM₁₀ reached the AQI “Good” level, PM_{2.5} was slightly higher than 12.1 μg/m³ and at the AQI “Moderate” level. The dust suppressing capability of tested 1-oxy melibioside is comparable to the suppression ability of Rhamnolipid C18 (FIG. 5). Furthermore, 1-oxy-melibioside C10 (Ls249M) suppressed dust greater than other glycolipid types with C10 single tails (FIGS. 5 and 7).

| | 1-oxy- | 1-oxy- | 1-oxy- |
|---|---|---|---|
| | melibioside C12 | melibioside C10 | melibioside C8 |
| PM₁₀ (μg/m³) | Good | Good | Good |
| PM_{2.5} (μg/m³) | Moderate | Moderate | Moderate |

### Cellobioside—Single Tail

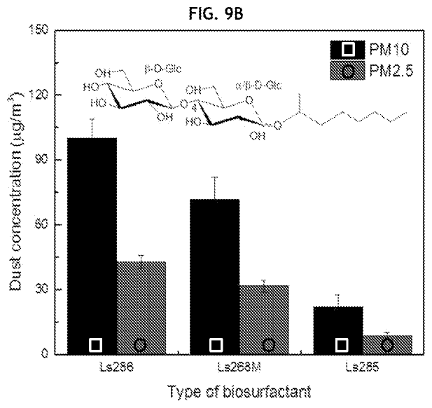

1-Oxy-cellobioside C8 and C10 effectively suppressed dust against 55 km/h wind and reached the AQI “Good” level for PM₁₀ and PM_{2.5} (FIG. 9A). Tested glycolipids with single tails generally reduced PM₁₀ and PM_{2.5} concentrations when increasing the length of single tails in glycolipids (FIGS. 5 and 7). However, this trend did not apply to 1-oxy melibioside C8-C12 and 1-oxy-cellobioside C12. 1-oxy melibioside C8-C12 maintained the PM₁₀ and PM_{2.5} concentrations, regardless of the tail length (FIG. 8). Unexpectedly, 1-oxy-cellobioside C12, compared to 1-oxy-cellobioside C8 and C10, did not suppress the dust and generated PM₁₀ and PM_{2.5} at the AQI “Hazardous” level (FIG. 9A). These results indicate that glycolipids with a single sugar head and a single tail can suppress the dust more effectively when the tail length is longer (FIGS. 5 and 9) but it is difficult to predict the dust suppressing trend with the length of the single tails when glycolipids have double sugar heads (FIGS. 8 and 9A). It is interesting to note that 1-oxy-cellobioside C10 (Ls261M; FIG. 9A) as well as 1-oxy-melibioside C10 (Ls249M; FIG. 8), which both have double sugar heads and C10 single tail, suppressed the dust greater than other glycolipid types with a single sugar head and C10 single tail (FIGS. 5 and 7) and reached to the AQI “Good” level.

| | 1-oxy- | 1-oxy- | 1-oxy- |
|---|---|---|---|
| | cellobioside C12 | cellobioside C10 | cellobioside C8 |
| PM₁₀ (μg/m³) | Hazardous | Good | Good |
| PM_{2.5} (μg/m³) | Hazardous | Good | Good |

### 2-Oxy-Cellobioside—Single Tail

Compared to 1-oxy-cellobioside C8 and C10 (FIG. 9A), the dust suppression of 2-oxy-cellobioside C8 and C10 were not as effective (FIG. 9B). However, 2-oxy-cellobioside C12, containing longer tails than other 2-oxy-cellobioside, effectively suppressed the dust against 55 km/h wind and reached the AQI “Good” level (FIG. 9B). 2-oxy-cellobioside follows the dust suppressing trend where the longer length of single tails in glycolipids reduces PM₁₀ and PM_{2.5} concentrations more effectively, although it contains double cellobiose sugar heads like 1-oxy-cellobioside.

| | 2-oxy- | 2-oxy- | 2-oxy- |
|---|---|---|---|
| | cellobioside C12 | cellobioside C10 | cellobioside C8 |
| PM₁₀ (μg/m³) | Good | Moderate | Moderate |
| PM_{2.5} (μg/m³) | Good | Moderate | Unhealthy for |
| | | | sensitive groups |

FIG. 10 shows the summary of dust suppression tests of various glycolipids.

Wettability of coal dust by glycolipids: Hydrophilic mine tailings dust can be suppressed by water until the water completely evaporates. To enhance the dust suppression ability, selected glycolipids were added to water, and their effectiveness as dust suppressants was evaluated after the water evaporation (FIG. 5-9B). In the case of hydrophobic dust (e.g., coal dust), however, water cannot be used alone since water does not wet hydrophobic materials. As a result, surfactants are necessary to wet and control hydrophobic dust pollution. Instead of using potentially toxic, conventional surfactants, bioinspired glycolipids that are sustainable, environmentally friendly, low toxic, and biodegradable were tested to investigate whether glycolipids can be a viable alternative for conventional surfactants. A sink test was conducted to identify types of glycolipids that can wet coal particles. The following glycolipids were evaluated in this test: Octyl β-D-glucopyranoside (Octyl), Xylolipid C10 (Xyl C10), Xylolipid C14 (Xyl C14), Xylolipid C18 (Xyl C18), Rhamnolipid C10C10 (Rha C10C10), Rhamnolipid C12C12 (Rha C12C12), Rhamnolipid C14C14 (Rha C14C14) and biosynthetic Dirhamnolipid (see FIGS. 2 and 3 for structures).

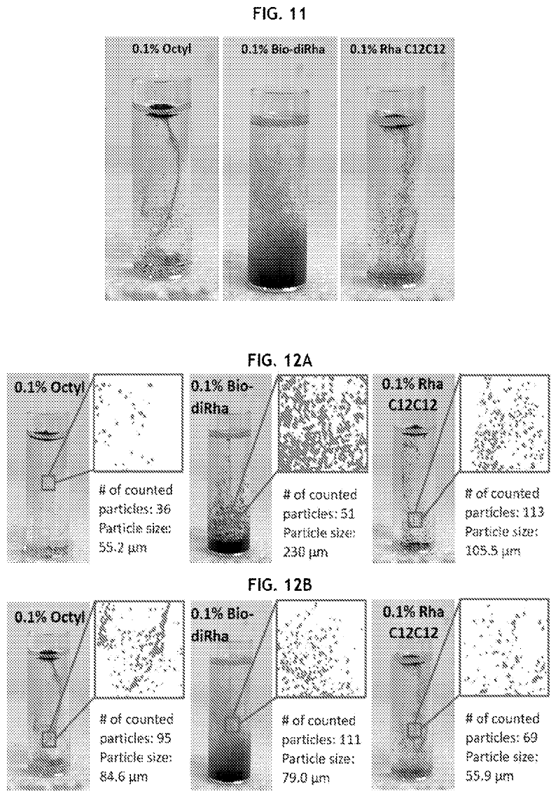

Except for the 0.1 w/v % Xyl C14 treatment, all tested glycolipids wet coal particles. Among them, biosynthetic Dirhamnolipid wet the hydrophobic particles the fastest, while Octyl and Rha C12C12 wet and settled the particles into the water the second fastest (FIG. 11).

Based on the recorded video of the sink test and visual inspection, there is no specific trend of coal wetting by glycolipids. Xyl C10 wet coal faster than Xyl C18, while Xyl C14 did not wet coal particles. Rha C12C12 wet coal, but RhaC10C10 and RhaC14C14 did not.

Coal dust binder: the formulation of glycolipids and HPMC: To identify the way to further improve hydrophobic dust wetting, a formulation was prepared by mixing 0.1 w/v % of glycolipids and 0.1 w/v % of amphiphilic cellulose, HPMC. Similar to the results from the sink test with only 0.1 w/v % glycolipids (FIG. 9), all tested solutions containing glycolipids together with HPMC wet coal particles, except the mixture of Xyl C14 and HPMC. Although most mixtures can wet hydrophobic dust, the major difference between the glycolipid solutions in the presence and absence of HPMC was the wetting speeds of coal particles. In general, when HPMC was introduced in glycolipid solutions, the wetting of coal particles was slower. However, the mixtures of HPMC with biosynthetic Dirhamnolipid or Rha C12C12 showed similar wetting speeds by visual inspection, whether or not HPMC was added. Therefore, in terms of coal particle wetting and wetting speeds, there seems no benefit to prepare the formulation or mixture of glycolipids and HPMC.

One distinct feature of solutions containing HPMC with biosynthetic Dirhamnolipid or Rha C12C12 (FIG. 11) was the presence of increased coal particle sizes. Since coal particles were sieved with a US standard 140 mesh (opening size: <100 μm), the particle sizes should be under 100 μm. However, the particle sizes were unexpectedly greater than 100 μm (upper particle size limit during sample preparation) in the solutions of HPMC with biosynthetic Dirhamnolipid or Rha C12C12 (FIG. 12). This suggests that HPMC with biosynthetic Dirhamnolipid or Rha C12C12 synergistically wet and agglomerated the hydrophobic coal particles, resulting in larger coal particle settling in the solutions, compared to other solutions. This data indicates that utilizing the formulation for coal dust sources can decrease the respirable, harmful ultrafine, and fine particles (PM_{2.5} and PM₁₀) by increasing their sizes.

Wetting and binding coal particles using other glycolipids: Wetting and/or agglomerating coal particles were performed using additional formulations comprising glycolipids only or together with HPMC. The list of tested glycolipids is as follows: Rhamnolipid (C10, C14, C18); Gal C18; 1-oxy-cellobioside (C8, C10, C12); 2-oxy-cellobioside (C8, C10, C12); 1-oxy-melibioside (C10, C12); Thio-glucoside; Thio-galactoside C10; Thio-cellobioside C10; Thio-maltibioside C10; Thio-lactoside C10.

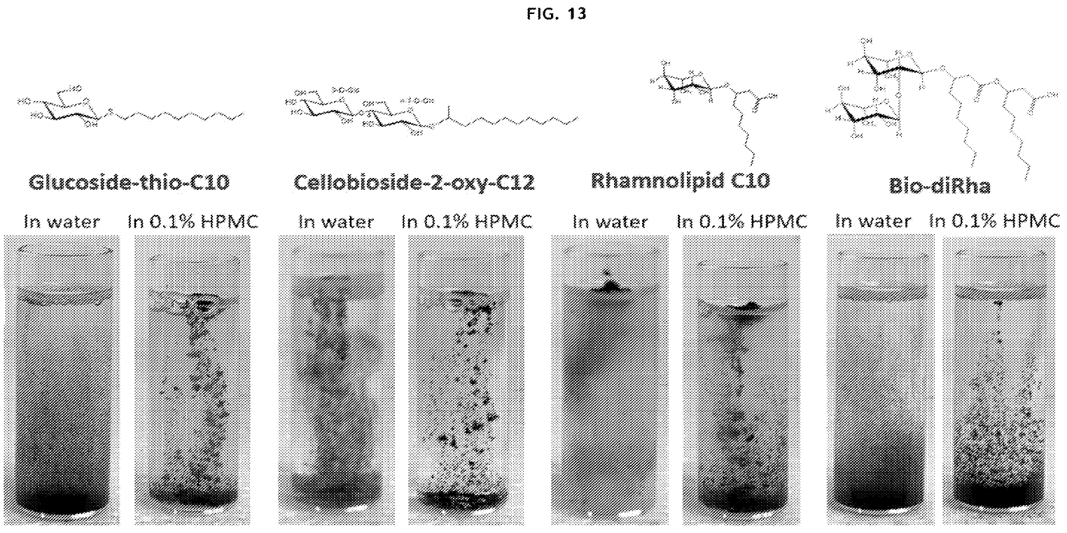

Among tested glycolipids in the presence and absence of HPMC, glucoside-thio-C10, cellobioside-2-oxy-C12, and rhamnolipid C10 were able to wet or wet and bind coal particles (FIG. 13). Interestingly, trends of wetting were identified with some of the glycolipids. For example, when comparing cellobioside-2-oxy-C8, -C10, and -C12, the glycolipids wet coal particles more rapidly when the C tail is longer. Contrastingly, shorter C tail length wet coal more rapidly when comparing rhamnolipid C10, C14 and C18. In addition to Bio-diRha (FIGS. 12 and 13), all three glycolipids (FIG. 13) also agglomerated coal particles. Therefore, it is concluded that the major benefit of using developed formulations containing both HPMC and glycolipids is not only the wetting of hydrophobic particles but also increasing particle sizes which decreases (ultra) fine or respirable dust. It is expected that implementing these formulations during the operation of the coal mining process or coal-fired power plant will potentially mitigate public health concerns related to respirable coal-related diseases, such as black-lung diseases or coal workers' pneumoconiosis.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method for suppressing dust from a substrate surface, airborne dust, or both, said method comprising applying an effective amount of a dust suppressant composition to an area, wherein said dust suppressant composition comprises an aqueous solution of a glycolipid.

2. (canceled)

3. The method of claim 1, wherein said glycolipid is according to the formula:
A-[B]ₐ
wherein a is 1 or 2; wherein A comprises a monosaccharide, a disaccharide, or a derivative thereof; and wherein B comprises a hydrogen or an alkyl group comprising 1 to 20 carbons.

4. (canceled)

5. The method of claim 3, wherein B is according to the formula 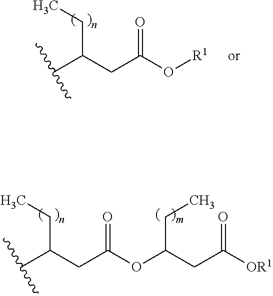
wherein each of m and n is independently an integer from 2 to about 20, and R¹ is H or C₁-C₂₀ alkyl.

6. The method of claim 3, wherein when a is 2, each B is attached to a different position of A.

7. The method of claim 3, wherein A is a monosaccharide or a thiol derivative thereof or an amine derivative thereof, wherein said monosaccharide is selected from the group consisting of glucose, galactose, rhamnose, arabinose, xylose, fucose, a thiol derivative thereof, and an amine derivative thereof.

8. (canceled)

9. The method of claim 7, wherein B is attached to the hydroxyl group of the anomeric carbon or a thiol derivative thereof or an amine derivative thereof of said monosaccharide.

10. The method of claim 3, wherein A is a disaccharide, a thiol derivative thereof, or an amine derivative thereof, wherein said disaccharide is selected from the group consisting of lactose, maltose, glucose, fructose, melibiose, cellobiose, rutinose, a thiol derivative thereof, and an amine derivative thereof.

11. (canceled)

12. The method of claim 10, wherein said disaccharide comprises 1→2, 1→4, or 1→6 linkage between two monosaccharides.

13. (canceled)

14. The method of claim 1, wherein said aqueous solution comprises about 10% w/v or less of said glycolipid, or wherein said aqueous solution comprises about 1% w/v or less of said glycolipid.

15. (canceled)

16. The method of claim 1, wherein said composition is applied at a rate of between 50,000 to 600,000 gallons per km².

17. The method of claim 1, wherein said dust suppressant composition further comprises alkylated cellulose.

18. The method of claim 17, wherein the alkylated cellulose comprises methyl cellulose, wherein the methyl cellulose comprises hydroxypropyl methyl cellulose.

19. (canceled)

20. The method of claim 1, wherein said substrate is mine tailings, soil, coal, a coal mine, dust, or a combination thereof.

21. A method for reducing formation of dust from a substrate surface, said method comprising applying a dust suppressant composition to said substrate surface, wherein said dust suppressant composition comprises an aqueous solution of a glycolipid of the formula:
A-[B]ₐ
wherein a is 1 or 2; A is selected from the group consisting of a monosaccharide, a disaccharide, and a derivative thereof; and B is a C6-C20 alkyl or a moiety of the formula: 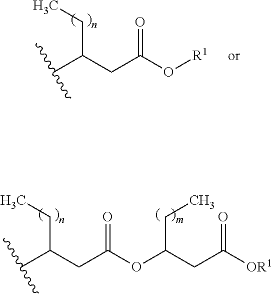
wherein each of m and n is independently an integer from 2 to about 20, and R¹ is H or C₁-C₂₀ alkyl.

22. (canceled)

23. The method of claim 21, wherein when a is 2, B is attached to a different position of A.

24. The method of claim 21, wherein A is selected from the group consisting of glucose, galactose, rhamnose, arabinose, xylose, fucose, lactose, maltose, glucose, fructose, melibiose, cellobiose, rutinose, a thiol derivative thereof, and an amine derivative thereof.

25. The method of claim 21, wherein said dust suppressant composition further comprises alkylated cellulose.

26. The method of claim 25, wherein the alkylated cellulose comprises methyl cellulose, wherein the methyl cellulose comprises hydroxypropyl methyl cellulose.

27. (canceled)

28. The method of claim 21, wherein said aqueous solution comprises about 10% w/v or less of said glycolipid, or wherein said aqueous solution comprises about 1% w/v or less of said glycolipid.

29.-30. (canceled)

31. The method of claim 21, wherein said substrate is mine tailings, soil, coal, a coal mine, dust, or a combination thereof.
